(19) United States
(12) Patent Application Publication
(10) Pub. No.: US 2025/0342925 A1
(43) Pub. Date: Nov. 6, 2025

(54) METHOD FOR PROVIDING FOOD ALLERGY TREATMENT SERVICE USING ORAL IMMUNOTHERAPY, COMPUTER PROGRAM STORED IN RECORDING MEDIUM TO IMPLEMENT METHOD, AND SYSTEM

(71) Applicants: SAMSUNG LIFE PUBLIC WELFARE FOUNDATION, Seoul (KR); WAYCEN INC., Seoul (KR); RESEARCH AND BUSINESS FOUNDATION SUNGKYUNKWAN UNIVERSITY, Suwon-si, Gyeonggi-do (KR)

(72) Inventors: Ji Hyun KIM, Seoul (KR); Kang Mo AHN, Seoul (KR); Kyung Nam KIM, Suwon-si, Gyeonggi-do (KR); Choong KI MIN, Seoul (KR); Jun Ho LEE, Incheon (KR); Daeun PARK, Seoul (KR)

(73) Assignees: SAMSUNG LIFE PUBLIC WELFARE FOUNDATION, Seoul (KR); WAYCEN INC., Seoul (KR); RESEARCH AND BUSINESS FOUNDATION SUNGKYUNKWAN UNIVERSITY, Suwon-si, Gyeonggi-do (KR)

(21) Appl. No.: 18/855,403

(22) PCT Filed: Apr. 17, 2023
(86) PCT No.: PCT/KR2023/005145

§ 371 Date: Oct. 9, 2024

(30) Foreign Application Priority Data
Apr. 15, 2022 (KR) 10-2022-0046870
Apr. 14, 2023 (KR) 10-2023-0049595

(51) Int. Cl. G16H 20/10; G06T 7/00; G10L 25/66; G16H 10/20; G16H 10/60; G16H 20/70; G16H 50/20; G16H 70/40; G16H 80/00

(52) U.S. Cl. CPC G16H 20/10; G06T 7/0012; G10L 25/66; G16H 10/20; G16H 10/60; G16H 20/70; G16H 50/20; G16H 70/40; G16H 80/00; G06T 2207/30004

(57) ABSTRACT

A method for providing a food allergy treatment service using oral immunotherapy, includes: transmitting patient information input into a patient terminal to a medical staff terminal; generating oral immunotherapy guide information on the basis of the patient information; transmitting the oral immunotherapy guide information to the patient terminal and displaying the same; when symptom information about symptoms after protein intake is input to the patient terminal, transmitting the symptom information to the medical staff terminal; analyzing the symptom information to determine an adverse reaction grade; and modifying the oral immunotherapy guide information, transmitting the same to the patient terminal, and displaying the oral immunotherapy guide information.

### CROSS-REFERENCE TO PRIOR APPLICATIONS

This Application is a National Stage Patent Application of PCT International Application No. PCT/KR2023/005145 (filed on Apr. 17, 2023), which claims priority to Korean Patent Application Nos. 10-2022-0046870 (filed on Apr. 15, 2022) and 10-2023-0049595 (filed on Apr. 14, 2023), which are all hereby incorporated by reference in their entirety.

### SUMMARY

The present invention relates to a food allergy treatment service provision method using oral immunotherapy, a computer program stored on a recording medium to implement the same, and a system for implementing the same, and more particularly to a food allergy treatment service provision method of managing a schedule for applying oral immunotherapy that safely and effectively induces desensitization in a patient with food allergy, a computer program stored on a recording medium to implement the same, and a system for implementing the same.

The prevalence of food allergies among pediatric age is 3 to 5%, and in 2015, a questionnaire epidemiological survey was conducted among elementary, middle, and high school students in Korea, and the prevalence of food allergy symptoms during their lifetime was 15.8%. The prevalence of anaphylaxis, a potentially fatal reaction, is also reported to be about 1%, accounting for 25 to 30% of all food allergies. The most common allergenic foods in children are eggs, milk, and wheat, which account for the majority of all culprit foods, and recent reports have shown that the natural remission of these allergies is delayed.

Eggs, milk, wheat, soybean, peanuts, and walnuts are not only a major source of nutrition for children, but they are also widely included in home-cooked and processed foods, which may lead to life-threatening incidents of unintentional intake of allergenic foods due to the implementation of congregate meals, despite the fact that dietary restrictions are often necessary even after entering school due to the delayed age of remission of food allergies. Furthermore, food allergies negatively impact children's psychological and physical behavioral development and emotional development through social experiences such as friendships, outdoor activities, and group life, leading to reports of parenting stress among parents of children with food allergies compared to parents of children without food allergies. In particular, the common triggers in the pediatric age group—eggs, milk, wheat, and soybean—are used in a wide variety of foods, making the process of dietary restriction very complex and may lead to nutritional imbalances.

In order to address these issues, oral immunotherapy has been studied in recent years, especially in the West, but no uniform protocols have been established. In addition, various oral immunotherapy programs have been implemented, but they are heterogeneous, raising the need for unified standards for the rate of escalation of food antigens, the amount of food antigen protein taken during maintenance, and the evaluation of adverse events. In particular, there is a need to address the problem of frequent allergic reactions during initial escalation of oral immunotherapy and frequent hospital visits due thereto, which may lead to low patient and guardian compliance and increased anxiety.

### SUMMARY

Therefore, it is an object of the present invention to provide a food allergy treatment service provision method using oral immunotherapy capable of reducing the number of hospital visits and reducing anxiety of patients and guardians by applying a uniform protocol to perform customized oral immunotherapy at home through a smartphone application, a computer program stored on a recording medium to implement the same, and a system for implementing the same.

In order to accomplish the above object, a food allergy treatment service provision method using oral immunotherapy according to the present invention includes the steps of (a) transmitting patient information input to a patient terminal to a medical staff terminal connected to the patient terminal over a network, (b) generating oral immunotherapy guideline information based on the patient information received by the medical staff terminal, (c) transmitting the oral immunotherapy guideline information to the patient terminal for display, (d) when symptom information about a symptom after protein intake is input to the patient terminal, transmitting the symptom information to the medical staff terminal, (e) analyzing the symptom information on the medical staff terminal to determine an adverse event grade, and (f) modifying the oral immunotherapy guideline information according to the adverse event grade and transmitting the modified oral immunotherapy guideline information to the patient terminal for display.

Step (b) may include determining a starting dose of oral immunotherapy with reference to the result of an oral food challenge (OFC) included in the patient information.

In addition, step (b) may be a step of generating the oral immunotherapy guideline information including a start date of an oral immunotherapy step, which is divided into initial escalation, buildup, and maintenance, and a food intake schedule including the amount of food corresponding to the amount of protein to be taken per day.

Here, the oral immunotherapy guideline information may include a food intake schedule in which the starting dose of oral immunotherapy is maintained for one to two weeks in the initial escalation step, the dose of oral immunotherapy is increased by 0.001 grams every week until a food antigen protein level of 0.01 grams is reached, the dose of oral immunotherapy is maintained for one to two weeks when a food antigen protein level of 0.01 grams is reached, the dose of oral immunotherapy is increased by 0.01 grams every week until a food antigen protein level of 0.05 grams is reached, the dose of oral immunotherapy is maintained for three to seven days when a food antigen protein level of 0.05 grams is reached, and the dose of oral immunotherapy is increased by 0.01 grams for three to seven days until a food antigen protein level of 0.1 grams is reached.

In addition, the oral immunotherapy guideline information may include a food intake schedule, which is one of a daily buildup schedule in which food is taken while a dose equivalent to 3 to 5% of the dose of the previous day is continuously increased every day until a food antigen protein level of 4 grams is reached in the buildup step, in which a food antigen protein level of 0.1 grams has been reached, and a weekly buildup schedule in which a dose equivalent to 15 to 25% of the dose of the previous week is increased, the dose is maintained for one week, and a dose equivalent to 15 to 25% of the maintained dose is taken the next week.

Step (c) may include daily displaying the amount of food corresponding to the amount of protein to be taken per day on the patient terminal every day according to a food intake schedule selected according to a selection command input to any one of the patient terminal and the medical staff terminal, which is one of the daily buildup schedule and the weekly buildup schedule.

The oral immunotherapy guideline information may include a message to visit a clinic since the oral food challenge is required in the maintenance step, in which a food antigen protein level of 4 grams has been reached.

Here, the oral immunotherapy guideline information may be generated so as to repeat the oral food challenge after 2 grams to 4 grams of food antigen protein is taken every day for 2 months if an adverse event occurs as the result of the oral food challenge.

Alternatively, the oral immunotherapy guideline information may be generated so as to take 4 grams of food antigen protein at least four times every week if no adverse event occurs as the result of the oral food challenge.

The patient information may include patient's age, gender, underlying diseases, medication, allergenic foods, past medical history, and the result of an oral food challenge (OFC).

The oral immunotherapy guideline information may be generated so as to include a food intake schedule including the amount of food corresponding to the amount of protein to be taken per day for each of the allergenic foods.

The symptom information may include text information recorded on the patient terminal, image information of the area where the symptom occurs, and voice information of recorded patient's voice.

Meanwhile, a computer program stored on a computer-readable storage medium includes a command configured to enable a processor to perform the steps of (a) transmitting patient information input to a patient terminal to a medical staff terminal connected to the patient terminal over a network, (b) generating oral immunotherapy guideline information based on the patient information received by the medical staff terminal, (c) transmitting the oral immunotherapy guideline information to the patient terminal for display, (d) when symptom information about a symptom after protein intake is input to the patient terminal, transmitting the symptom information to the medical staff terminal, (e) analyzing the symptom information on the medical staff terminal to determine an adverse event grade, and (f) modifying the oral immunotherapy guideline information according to the adverse event grade and transmitting the modified oral immunotherapy guideline information to the patient terminal for display.

Here, the patient information may include at least one of a psychological questionnaire for a patient, a psychological questionnaire for a guardian, presence or absence of specific IgE on a blood test of the patient, and the result of analysis of microbiome.

The timing and cycle of buildup in the oral immunotherapy guideline information may be changed based on the result of at least one of the psychological questionnaire of the patient and the psychological questionnaire of the guardian.

Each of the psychological questionnaire of the patient and the psychological questionnaire of the guardian may include at least one of an anxiety and quality of life assessment tool, a patient health questionnaire, and a resilience scale test.

Mindset content for psychological reassurance may be provided based on the result of at least one of the psychological questionnaire of the patient and the psychological questionnaire of the guardian.

The mindset content may include psychological counseling and educational videos on oral immunotherapy.

A step of collecting, by the patient terminal, data about patients having similar symptoms based on symptom information of a patient input thereto and a buildup step may be further included, wherein the data may include an encouragement message and a review of treatment outcome.

A step of providing a supplementary therapy of strain based on the result of analysis of the microbiome may be further included.

A food allergy treatment service provision method using oral immunotherapy according to the present invention includes the steps of (a) conducting a first survey to check a psychological state of a patient through a patient terminal and a medical staff terminal connected to the patient terminal over a network, (b) generating oral immunotherapy guideline information based on patient information registered in the medical staff terminal and transmitting the generated oral immunotherapy guideline information to the patient terminal during initial escalation, (c) upon determining that at least one of anxiety and depression is equal to or greater than a reference value from the result of analysis of the first survey on the medical staff terminal, modifying the oral immunotherapy guideline information so as to take the same dose of protein for a predetermined period of time and transmitting the modified oral immunotherapy guideline information to the patient terminal, (d) conducting a second survey to check the psychological state through the patient terminal and the medical staff terminal, and (e) upon determining that the psychological state has not improved from the result of analysis of the second survey on the medical staff terminal, modifying the oral immunotherapy guideline information and transmitting the modified oral immunotherapy guideline information to the patient terminal during buildup.

The food allergy treatment service provision method may further include a step of inputting the result of a blood and gut microbiome test to the patient terminal and transmitting the result to the medical staff terminal, wherein step (e) may be further analyzing the result of the blood and gut microbiome test to determine whether the psychological state has improved.

Step (e) may be a step of transmitting, to the patient terminal during buildup, the oral immunotherapy guideline information modified so as to alternately perform a daily buildup schedule in which food is taken while a dose equivalent to 3 to 5% of the dose of the previous day is continuously increased every day until a food antigen protein level of 4 grams is reached in the buildup step, in which a food antigen protein level of 0.1 grams has been reached, and a weekly buildup schedule in which a dose equivalent to 15 to 25% of the dose of the previous week is increased, the dose is maintained for one week, and a dose equivalent to 15 to 25% of the maintained dose is taken the next week.

Alternatively, step (e) may be a step of transmitting, to the patient terminal during buildup, the oral immunotherapy guideline information modified through any one of a change to less than 5% if a daily buildup schedule is set such that food is taken while a dose equivalent to 5% of the dose of the previous day is continuously increased every day until a food antigen protein level of 4 grams is reached, in the buildup step, in which a food antigen protein level of 0.1 grams has been reached, and a change to less than 25% if a weekly buildup schedule is set to increase the dose by 25% from the previous week until a food antigen protein level of 4 grams is reached.

Meanwhile, a computer program stored on a computer-readable storage medium includes a command configured to enable a processor to perform the steps of (a) conducting a first survey to check a psychological state of a patient through a patient terminal and a medical staff terminal connected to the patient terminal over a network, (b) generating oral immunotherapy guideline information based on patient information registered in the medical staff terminal and transmitting the generated oral immunotherapy guideline information to the patient terminal during initial escalation, (c) upon determining that at least one of anxiety and depression is equal to or greater than a reference value from the result of analysis of the first survey on the medical staff terminal, modifying the oral immunotherapy guideline information so as to take the same dose of protein for a predetermined period of time and transmitting the modified oral immunotherapy guideline information to the patient terminal, (d) conducting survey to check the psychological state through the patient terminal and the medical staff terminal, and (e) upon determining that the psychological state has not improved from the result of analysis of the second survey on the medical staff terminal, modifying the oral immunotherapy guideline information and transmitting the modified oral immunotherapy guideline information to the patient terminal during buildup.

A food allergy treatment service provision system using oral immunotherapy according to the present invention includes a patient terminal configured to allow patient information to be input thereto and a medical staff terminal configured to receive the patient information transmitted from the patient terminal and generating oral immunotherapy guideline information, wherein the patient terminal displays the oral immunotherapy guideline information and allows symptom information about a symptom after protein intake to be input thereto, and the medical staff terminal receives the symptom information transmitted from the patient terminal, analyzes the received symptom information to determine an adverse event grade, modifies the oral immunotherapy guideline information according to the adverse event grade, and transmits the modified oral immunotherapy guideline information to the patient terminal.

In addition, the patient terminal may display question information for first and second surveys to check the psychological state of a patient and a guardian, and, when answer information for the first and second surveys is input, transmit the input answer information to the medical staff terminal, and the medical staff terminal may generate the question information for the first and second surveys, transmit the generated question information to the patient terminal, analyze the answer information transmitted from the patient terminal, modify the oral immunotherapy guideline information according to the degree of anxiety or depression of the patient and the guardian, and transmit the modified oral immunotherapy guideline information to the patient terminal.

According to a food allergy treatment service provision method using oral immunotherapy of the present invention, a computer program stored on a recording medium to implement the same, and a system for implementing the same, it is possible to safely and effectively conduct oral immunotherapy at home by controlling food intake and monitoring side effects at home using an application.

In addition, it is possible to conduct oral immunotherapy in a psychologically comfortable manner by dramatically reducing the frequency of allergic reactions and adverse events during initial escalation, and it is possible to reduce the number of hospital visits, to improve quality of life, and to reduce medical care costs through development of a food allergy treatment service provision system capable of achieving underlying immune tolerance.

Oral immunotherapy may be initiated in large hospitals under the supervision of a specialist and continued at home or in primary health clinics using an automated food allergy treatment service provision system.

It is possible to provide individually customized oral immunotherapy guideline information that reflects the characteristics of individuals, including psychological and psychiatric characteristics of food allergy patients and their guardians.

It is possible to transmit allergic symptoms that occur during oral immunotherapy in the form of images and voice, to automatically read allergic symptoms, and to transmit a message to address an adverse event, thereby ensuring efficient and safe continuation of oral immunotherapy.

It is possible to reduce school lunch-related deaths and to ensure a safe school day, thereby reducing mental stress and preventing decline in quality of life.

### BRIEF DESCRIPTION OF THE DRAWINGS

FIG. 1 is a block diagram showing the configuration of a food allergy treatment service provision system using oral immunotherapy according to an embodiment of the present invention;

FIGS. 2A to 2D are views describing the operation of a patient application executed on a patient terminal included in the food allergy treatment service provision system of FIG. 1;

FIGS. 3A and 3B are views describing the operation of a medical staff console executed on a medical staff terminal included in the food allergy treatment service provision system of FIG. 1;

FIG. 4 is a flowchart describing a food allergy treatment service provision method using oral immunotherapy according to an embodiment of the present invention;

FIG. 5 is a graph illustrating an oral immunotherapy step in the food allergy treatment service provision method using oral immunotherapy according to the embodiment of the present invention; and

FIG. 6 is a flowchart describing a food allergy treatment service provision method using oral immunotherapy according to another embodiment of the present invention.

### DETAILED DESCRIPTION

Hereinafter, a food allergy treatment service provision method using oral immunotherapy according to a preferred embodiment of the present invention, a computer program stored on a recording medium for implementing the same, and a system (hereinafter referred to as a “food allergy treatment service method,” a “computer program,” and a “food allergy treatment service provision system”) will be described in detail with reference to the accompanying drawings.

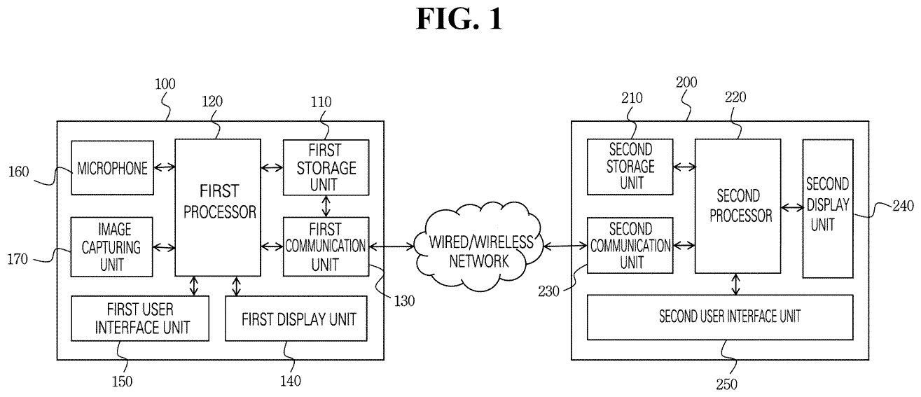

FIG. 1 is a block diagram showing the configuration of a food allergy treatment service provision system using oral immunotherapy according to an embodiment of the present invention. Referring to FIG. 1, the food allergy treatment service provision system of the present invention includes a patient terminal 100 and a medical staff terminal 200, wherein the patient terminal 100 and the medical staff terminal 200 are connected to each other over a wired/wireless network to transmit and receive signals, data, information, and the like.

A wired/wireless network commonly refers to a communication network that supports various communication standards or protocols for pairing or data transmission and reception between the patient terminal 100 and the medical staff terminal 200. The wired/wireless network may include any communication network that is currently or will be supported through standardization, and may support one or more communication protocols therefor.

The patient terminal 100 may be implemented by a mobile terminal, such as a smartphone, a tablet PC, or an ultrabook. However, the present invention is not limited thereto, and the patient terminal may also include a stationary device, such as a personal computer, a microprocessor, a mainframe computer, a digital processor, or a device controller.

The patient terminal 100 includes a first storage unit 110, a first processor 120, a first communication unit 130, a first display unit 140, a first user interface unit 150, a microphone 160, and an image capturing unit 170. Each component may be integrated, added, or omitted depending on the specifications of the patient terminal 100 that is actually implemented. That is, two or more components may be combined into one component, or one component may be subdivided into two or more components, as needed. In addition, the functions performed in each block are for the purpose of describing the embodiment of the present invention, and specific operations or devices are not intended to limit the scope of the present invention.

The first storage unit 110 may store data supporting various functions of the patient terminal 100. That is, the first storage unit 110 may store a plurality of application programs executed on the patient terminal 100, data for operation of the patient terminal 100, commands, and at least one program command. At least some of the application programs may be present on the patient terminal 100 from the factory for basic functions of the patient terminal 100, or may be downloaded from an external server through wired/wireless communication.

In the present invention, the first storage unit 110 stores a patient application, among computer programs of the present invention, and stores oral immunotherapy guideline information transmitted from the medical staff terminal 200. In addition, the first storage unit 110 stores information about the amount of protein actually consumed by a patient, the date and time of intake, and the symptoms after intake. Furthermore, if the patient takes medication or receives injection treatment, medication history information about the type and dose of the medication administered is also stored.

The first processor 120 may control the overall operation of the patient terminal 100, and may provide or process appropriate information or functions by processing signals, data, information, etc. input or output through the components of the patient terminal 100 or by executing the application programs stored in the first storage unit 110. In addition, the first processor 120 may control at least some of the components of the patient terminal 100 in order to execute the application programs stored in the first storage unit 110. Furthermore, the first processor 120 may operate at least two of the components included in the patient terminal 100 in combination in order to execute the application programs.

The first processor 120 may be constituted by one or more cores, and may be any of a variety of commercially available processors. For example, the first processor 120 may include a central processing unit (CPU), a general purpose graphics processing unit (GPGPU), and a tensor processing unit (TPU). In addition, the first processor 120 may be constituted by a dual processor or a multiprocessor architecture.

In the present invention, the first processor 120 executes the patient application stored in the first storage unit 110 to display the oral immunotherapy guideline information through the first display unit 140. In addition, when patient information and symptom information about the symptoms after the patient actually takes the protein are input through the first user interface unit 150, the first processor 120 transmits the information to the medical staff terminal 200 through the first communication unit 130. The first processor 120 controls the first display unit 140 to display the oral immunotherapy guideline information transmitted by the medical staff terminal 200, and controls data transmission and reception to and from the medical staff terminal 200 to enable the patient to chat and inquire with the medical staff 24 hours a day through the patient application.

The first processor 120 displays question information of first and second surveys for checking the mental state of the patient and a guardian through the first display unit 140, and, when answer information of the first and second surveys is input through the first user interface unit 150, transmits the information to the medical staff terminal 200 through the first communication unit 130.

The first communication unit 130 may include one or more modules configured to enable wired/wireless communication between the patient terminal 100 and the wired/wireless network, between the patient terminal 100 and another device, or between the patient terminal 100 and an external server. In addition, the first communication unit 130 may include one or more modules configured to connect the patient terminal 100 to one or more networks. The first communication unit 130 refers to a module for wired/wireless network connection, which may be built into or outside the patient terminal 100 and may be configured to transmit and receive wired/wireless signals.

In the present invention, the patient application, among the oral immunotherapy management programs, is downloaded from the external server through the first communication unit 130. In addition, the oral immunotherapy guideline information is received from the medical staff terminal 200 through the first communication unit 130, and the information about the amount of protein taken by the patient, the date and time of intake, and the symptoms after intake and the medication history information about the type and dose of the medication administered input through the first user interface unit 150 are transmitted to the medical staff terminal 200.

The first display unit 140 displays various application programs executed on the patient terminal 100 on the screen. The oral immunotherapy guideline information is provided to the patient through the patient application of the present invention displayed on the first display unit 140. That is, the protein intake amount and the intake date according to oral immunotherapy customized for each patient are displayed through the first display unit 140, and a corresponding method according to the symptoms is also displayed through the first display unit 140.

The first user interface unit 150 is constituted by a keyboard, a mouse, and a touch panel, and receives a user command for controlling the operation of the patient terminal 100. In the present invention, the patient information, the amount of protein actually consumed by the patient, and the date and time of intake are input through the first user interface unit 150, and symptom information about the symptoms after intake is input through the first user interface unit 150.

The microphone 160, which is a means configured to record user voice, records patient voice when respiratory symptoms occur after protein intake, and recorded voice information is transmitted to the medical staff terminal 200 through the first communication unit 130.

The image capturing unit 170 captures an image of the skin condition of the patient when skin symptoms such as hives occur image information is after protein intake, and captured transmitted to the medical staff terminal 200 through the first communication unit 130.

Meanwhile, the medical staff terminal 200 of the present invention may be paired with or connected to the patient terminal 100 over the wired/wireless network to transmit, receive, and display predetermined data. The medical staff terminal 200 includes a second storage unit 210, a second processor 220, a second communication unit 230, a second display unit 240, and a second user interface unit 250. Each component may be integrated, added, or omitted depending on the specifications of the medical staff terminal 200 that is actually implemented. That is, two or more components may be combined into one component, or one component may be subdivided into two or more components, as needed. In addition, the functions performed in each block are for the purpose of describing the embodiment of the present invention, and specific operations or devices are not intended to limit the scope of the present invention.

The second storage unit 210 may store data supporting various functions of the medical staff terminal 200. That is, second storage unit 210 may store a plurality of application programs executed on the medical staff terminal 200, data for operation of the medical staff terminal 200, commands, and at least one program command. At least some of the application programs may be present on the medical staff terminal 200 from the factory for basic functions of the medical staff terminal 200, or may be downloaded from the external server through wired/wireless communication. In the present invention, the second storage unit 210 stores a medical staff console, among the computer programs of the present invention.

The second processor 220 may control the overall operation of the medical staff terminal 200, and may provide or process appropriate information or functions by processing signals, data, information, etc. input or output through the components of the medical staff terminal 200 or by executing the application programs stored in the second storage unit 210. In addition, the second processor 220 may control at least some of the components of the medical staff terminal 200 in order to execute the application programs stored in the second storage unit 210. Furthermore, the second processor 220 may operate at least two of the components included in the medical staff terminal 200 in combination in order to execute the application programs.

The second processor 220 may be constituted by one or more cores, and may be any of a variety of commercially available processors. For example, the second processor 220 may include a central processing unit (CPU), a general purpose graphics processing unit (GPGPU), and a tensor processing unit (TPU). In addition, the second processor 220 may be constituted by a dual processor or a multiprocessor architecture.

In the present invention, the second processor 220 executes the medical staff console stored in the second storage unit 210 to register patient information and to generate oral immunotherapy guideline information customized for each patient based on the registered patient information. In addition, the second processor 220 analyzes symptom information transmitted from the patient terminal 100 to determine an adverse event grade, modifies the oral immunotherapy guideline information based on the adverse event grade, and transmits the modified oral immunotherapy guideline information to the patient terminal 100. The second processor 220 controls data transmission and reception to and from the patient terminal 100 to enable the medical staff to chat and consult with the patient 24 hours a day.

The second processor 220 generates question information for first and second surveys to check the psychological state of the patient and the guardian and transmits the generated question information to the patient terminal 100 through the second communication unit 230. The second processor analyzes answer information transmitted from the patient terminal 100 to determine the degree of anxiety or depression of the patient and the guardian, modifies the oral immunotherapy guideline information, and transmits the modified oral immunotherapy guideline information to the patient terminal 100 through the second communication unit 230.

The second communication unit 230 may include one or more modules configured to enable wired/wireless communication between the medical staff terminal 200 and the wired/wireless network, between the medical staff terminal 200 and another device, or between the medical staff terminal 200 and the external server. In addition, the second communication unit 230 may include one or more modules configured to connect the medical staff terminal 200 to one or more networks. The second communication unit 230 refers to a module for wired/wireless network connection, which may be built into or outside the medical staff terminal 200 and may be configured to transmit and receive wired/wireless signals.

In the present invention, the information about the amount of protein taken by the patient, the date and time of intake, and the symptoms after intake and the medication history information about the type and dose of the medication administered are received from the patient terminal 100, and the oral immunotherapy guideline information is transmitted to the patient terminal 100, through the second communication unit 230.

The second display unit 240 displays various application programs executed on the medical staff terminal 200 on the screen. The medical staff may check the amount of protein actually consumed by the patient and the date and time of intake through the medical staff console of the present invention displayed on the second display unit 240, and may also check the symptoms after intake.

The second user interface unit 250 is constituted by a keyboard, a mouse, and a touch panel, and receives a user command for controlling the operation of the medical staff terminal 200. In the present invention, patient information is registered and modified, and a medical staff command for creating or modifying the oral immunotherapy guideline information is input, through the second user interface unit 250

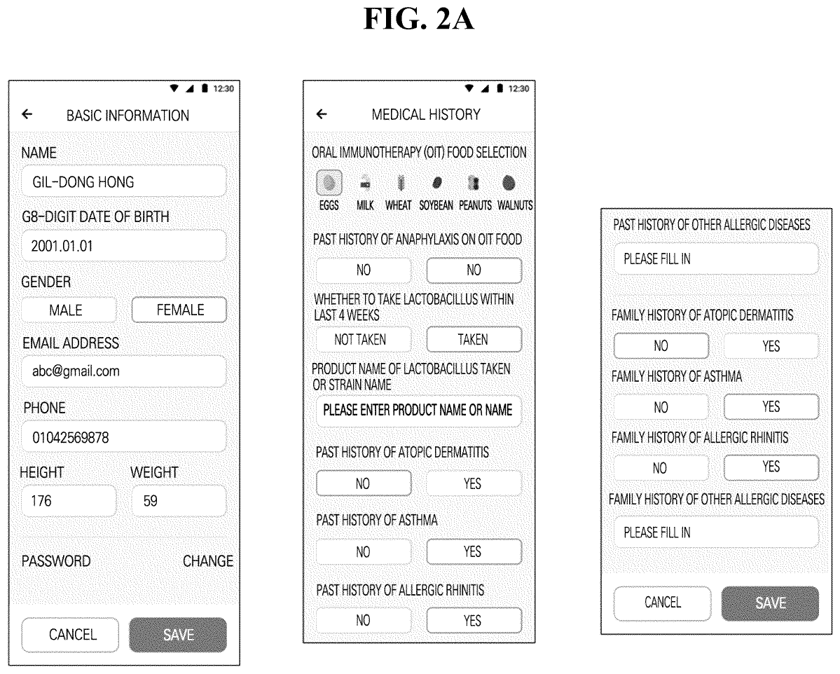

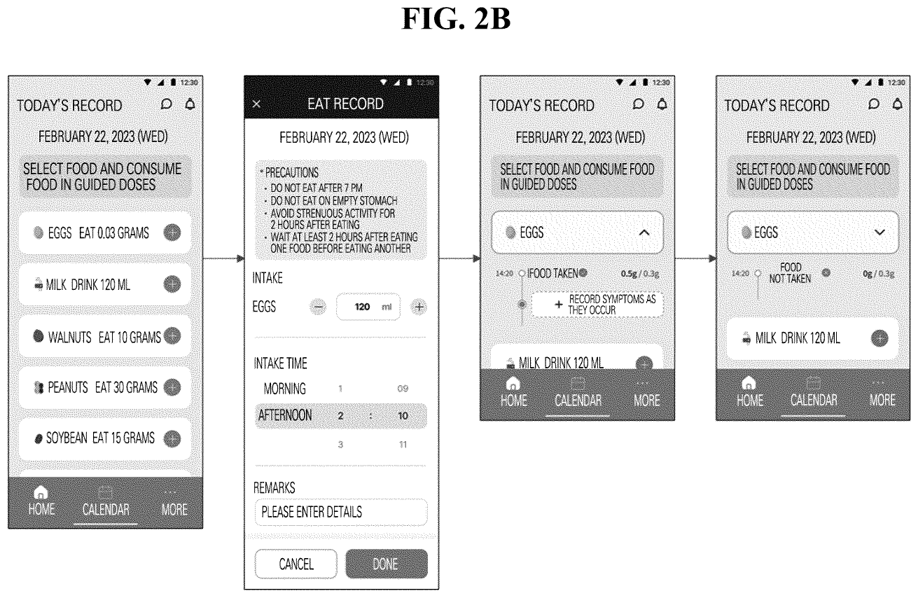

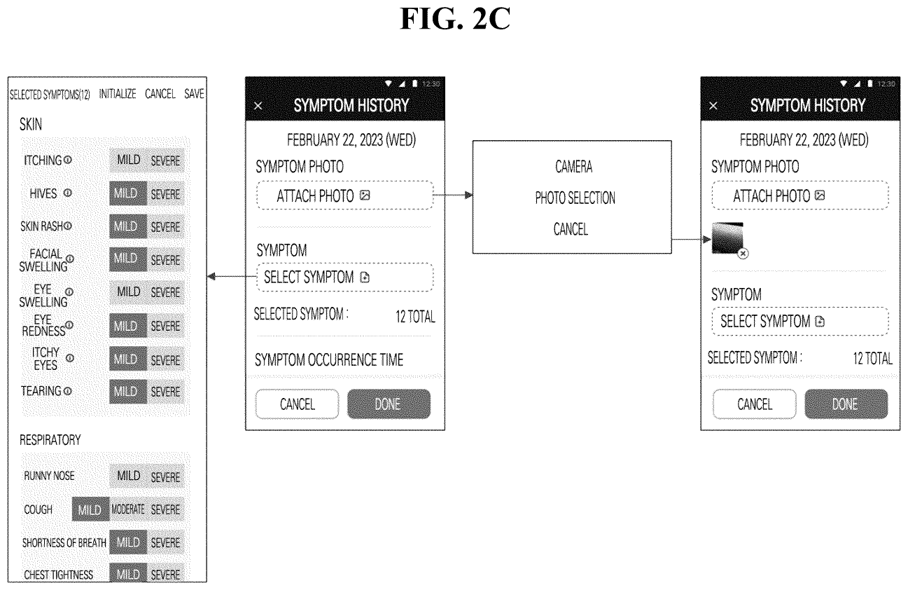

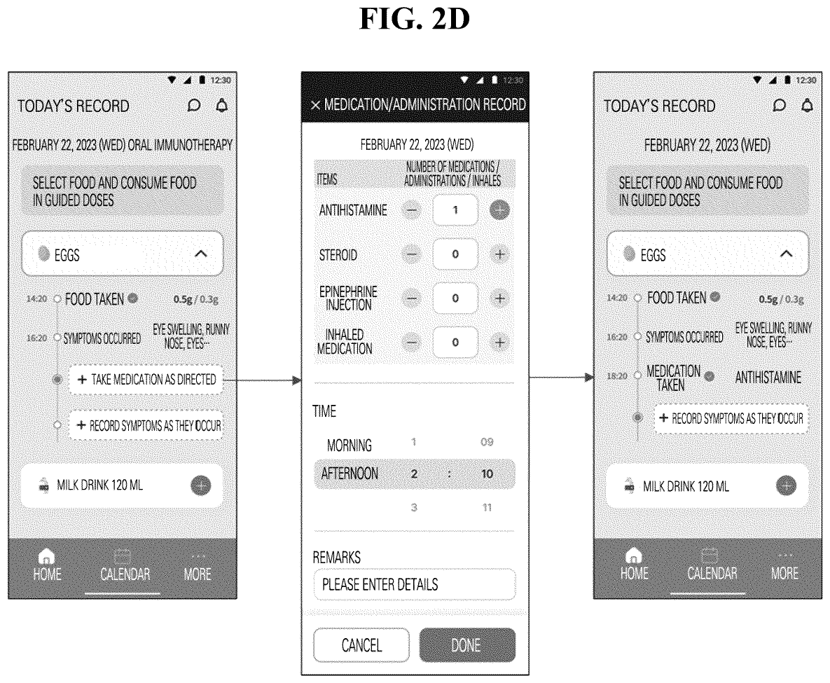

FIGS. 2A to 2D are views describing the operation of the patient application executed on the patient terminal included in the food allergy treatment service provision system of FIG. 1. FIG. 2A is a view illustrating a process of registering patient information through the patient application executed on the patient terminal 100. Through the patient application, the patient may register basic information, such as the patient's name, age, gender, underlying diseases, medication, allergenic foods, and past history of allergic diseases, as patient information, and may also register the results of the oral food challenge (OFC) as patient information. The patient information is transmitted to the medical staff terminal 200, and when the medical staff approves the registration of the patient information, customized oral immunotherapy guideline information is generated based on the patient information.

FIGS. 2B to 2D are views illustrating a process of performing a food allergy treatment service provision method using oral immunotherapy through the patient application executed on the patient terminal 100.

As shown in FIG. 2B, oral immunotherapy guideline information about the intake dose and precautions of food antigen protein is provided, and the patient clicks a ‘+’ button to record the date and time of intake of food antigen protein and the amount of intake. The date and time of intake of the protein and the amount of intake are stored in the patient terminal 100 and are transmitted to the medical staff terminal 200. If each food is selected on today's record, whether the food has been taken may be checked, and if the date and time of protein intake and the amount of intake are not received by the medical staff terminal 200 within a predetermined time, the medical staff terminal 200 generates an alarm to take the food antigen protein and to record the date and time of intake and the amount of intake, and the alarm is transmitted to the patient terminal 100.

As shown in FIG. 2C, the symptoms after the protein intake and the time at which the symptoms occurred are recorded, and if skin symptoms such as hives occur, an image of the area is captured, and if respiratory symptoms occur, voice is recorded. Symptom information about the symptoms after intake of food antigen protein, such as text information recorded about the symptoms, the image information of the area where the symptoms occurred, voice information of the recorded patient's voice, and the time at which the symptoms occurred, is stored in the patient terminal 100 and is transmitted to the medical staff terminal 200.

The medical staff terminal 200 analyzes the received symptom information to determine the adverse event grade. The symptom categories for determining the adverse event grade are divided into a skin category, which includes symptoms of itching, hives, skin rash, and facial swelling; a conjunctival category, which includes symptoms of eye redness, itching, and watery eyes; an upper respiratory tract category, which includes symptoms of runny nose and mild cough; a lower respiratory tract category, which includes symptoms of moderate or severe cough, shortness of breath, chest tightness, wheezing, and choking; a digestive system category, which includes symptoms of nausea, abdominal pain, vomiting, and diarrhea; and a circulatory system category, which includes symptoms of decreased blood pressure, decreased consciousness, and cyanosis.

Adverse events are graded on a scale of mild-moderate-severe, or the consortium of food allergy research (CoFAR) scale of five. CoFAR Grade 1 is the case in which symptoms in one of the skin, conjunctival, upper respiratory tract, and digestive system categories are all mild. CoFAR Grade 2 is the case in which even any one of the symptoms in one of the skin, conjunctival, upper respiratory tract, and digestive system categories is severe or the case in which the symptoms in two or more categories are all mild. CoFAR Grade 3 is the case in which severe abdominal pain occurs or vomiting/diarrhea occurs three or more times or the case in which the symptoms in the lower respiratory tract category are all mild. In this case, the cough is moderate or severe. CoFAR Grade 4 is the case in which any one of the symptoms in the lower respiratory tract category is severe or the case in which any one of the symptoms in the circulatory system category occurs. CoFAR Grade 5 is fatal. CoFAR grade 1 may be categorized as mild, CoFAR grade 2 as moderate, and CoFAR grade 3 or higher as severe, or CoFAR grade 1 as mild, CoFAR grades 2 and 3 as moderate, and CoFAR grade 4 or higher as severe.

If a severe (CoFAR 3 or higher) or moderate (CoFAR 2) case is determined to have occurred twice or more within a month, a warning message indicating that a consultation with the medical staff is required is output through the patient terminal 100. If it is determined that the case is mild (CoFAR grade 1) or moderate (CoFAR grade 2), as illustrated in FIG. 2D, a medication guideline message is output, and if medication history information about the type and dose of the medication administered after intake of the medication is input to the patient terminal 100, the medication history information is transmitted to the medical staff terminal 200. Accordingly, the modified oral immunotherapy guideline information is transmitted from the medical staff terminal 200 to the patient terminal 100, on which the modified oral immunotherapy guideline information is displayed.

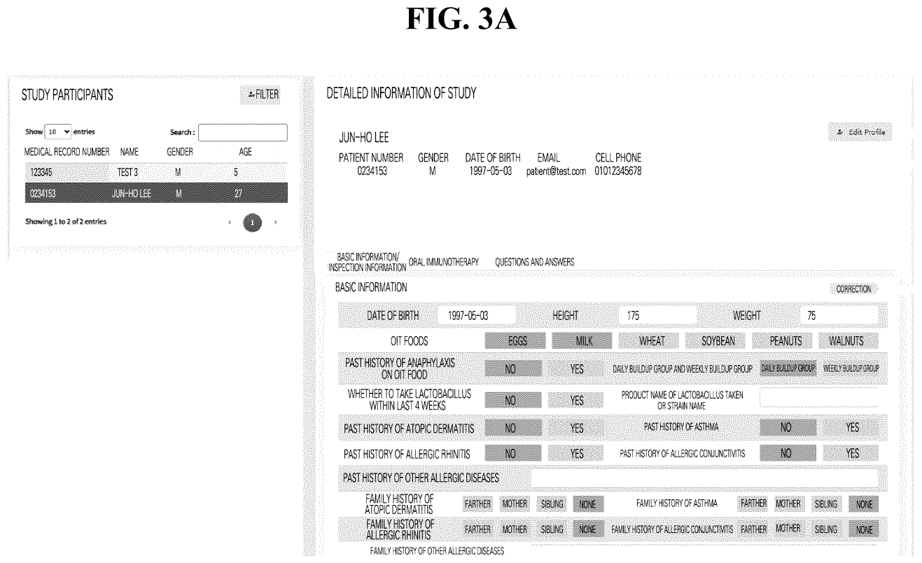

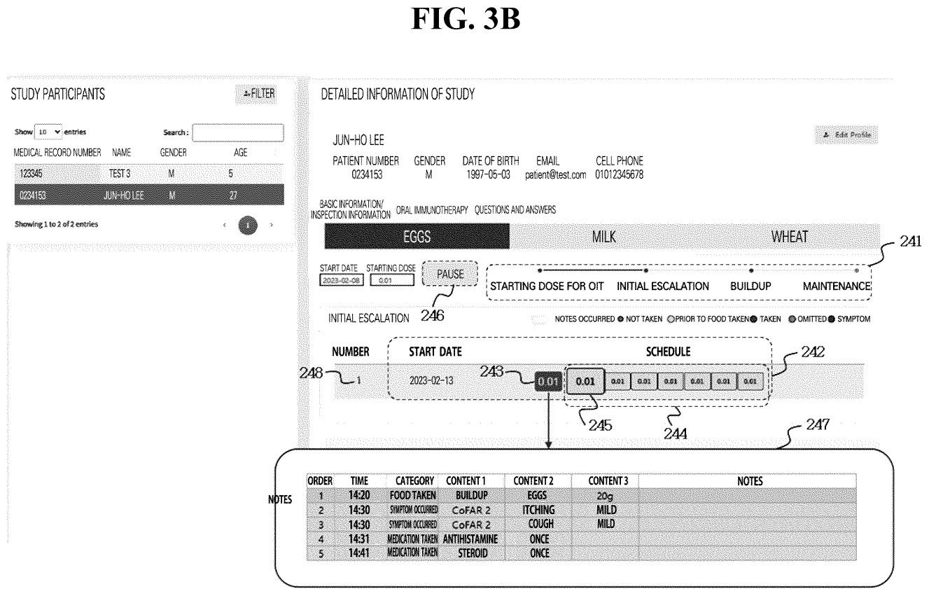

FIGS. 3A and 3B are views describing the operation of the medical staff console executed on the medical staff terminal 200 included in the food allergy treatment service provision system of FIG. 1.

FIG. 3A is a view illustrating a screen for checking the patient information transmitted from the patient terminal 100 through the medical staff console executed on the medical staff terminal 200. The medical staff may check and modify the patient information through the medical staff console. Customized oral immunotherapy guideline information is generated based on the registered patient information, as shown in FIG. 3B. The medical staff may generate and modify the oral immunotherapy guideline information through the medical staff console.

As illustrated in FIG. 3A, if eggs and milk are selected as the foods to which patient is allergic, oral immunotherapy guideline information including a food intake schedule for each of the eggs and the milk is generated. Referring to FIG. 3B, if the starting dose of oral immunotherapy for egg protein is determined and the start date is determined with reference to the results of oral food challenge in the patient information, a food intake schedule 241 is generated in an oral immunotherapy step 242, which is divided into initial escalation, buildup, and maintenance.

The initial escalation is a step in which the food antigen protein is gradually increased by 0.001 to 0.01 grams every 3 to 7 days until 0.1 grams of food antigen protein is reached. The buildup is a step in which the food antigen protein is increased by 5% every day or by 25% every week until 4 grams of food antigen protein is reached from 0.1 grams of food antigen protein. The maintenance is a step in which an amount equivalent to 4 grams of food antigen protein is taken once a day or at least four times every week. Although not illustrated in FIG. 3B, dietary restriction in which protein intake is restricted may be further included after the maintenance.

In the food intake schedule 242 of the initial escalation illustrated in FIG. 3B, the start date of the initial escalation and the amount of food antigen protein to be taken per day are displayed in eight slots 243. The schedule may be modified by deleting the current slot 245 selected from among intake schedule slots 244 or by adding a new slot to the left of the current slot 245. The food intake schedule 242 thus generated is displayed through the patient application executed on the patient terminal 100, as illustrated in FIG. 2B.

As illustrated in FIGS. 2B to 2D, the date and time of intake, the amount of intake, the symptom information about the symptoms after intake, and the medication history information about the type and dose of the medication administered are input to the patient terminal 100, are transmitted to the medical staff terminal 200, and are displayed through a pop-up window 247 in FIG. 3B. If the medical staff terminal 200 analyzes the symptom information and determines that the adverse event grade is moderate (CoFAR grade 2), the oral immunotherapy is paused (246), as illustrated in FIG. 3B.

If the pause 246 is clicked to resume the oral immunotherapy, the oral immunotherapy proceeds from the current paused slot 245. If the restart date is changed, it is restarted from the paused slot 245 on the changed date, and if the restarting dose is changed, a new step is added under the current step 248 to restart from the start date.

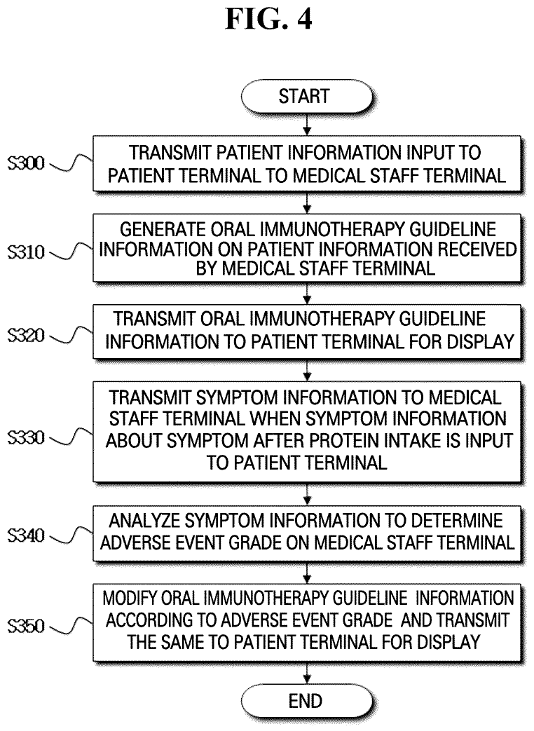

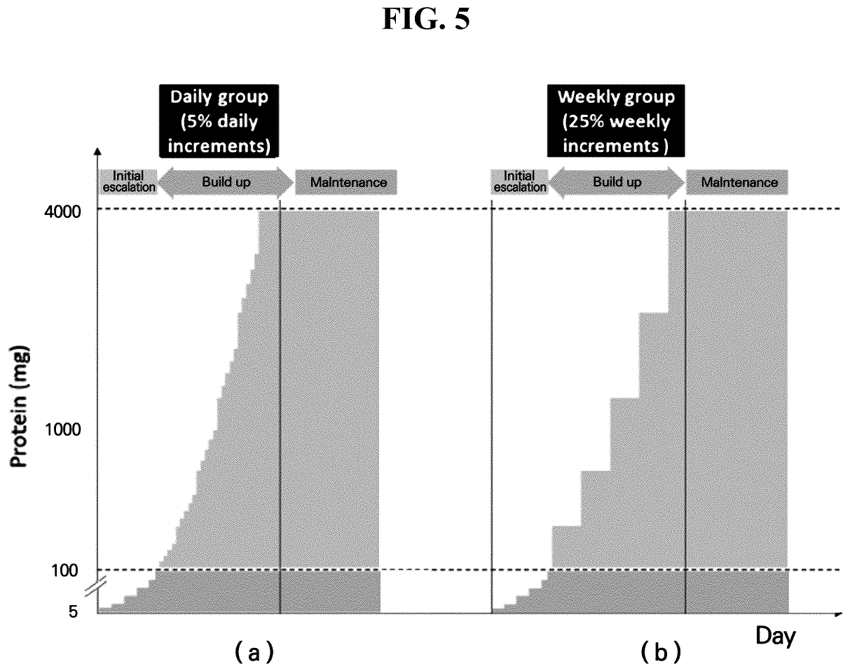

FIG. 4 is a flowchart describing a food allergy treatment service provision method using oral immunotherapy according to an embodiment of the present invention, and FIG. 5 is a graph illustrating an oral immunotherapy step in the food allergy treatment service provision method using oral immunotherapy according to the embodiment of the present invention.

Referring to FIG. 4, first, the patient information input to the patient terminal 100 is transmitted to the medical staff terminal 200 (S300). The patient information includes at least one of the patient's age, gender, underlying diseases, medication, allergenic foods, past medical history, and oral food challenge (OFC) results.

Oral immunotherapy guideline information is generated based on the patient information received by the medical staff terminal 200 (S310). At this time, the oral immunotherapy guideline information is generated so as to include a food intake schedule including the amount of food corresponding to the amount of protein to be taken per day for each of the allergenic foods. For example, for a patient who is allergic to eggs and milk, among eggs, milk, wheat, peanuts, and walnuts, oral immunotherapy guideline information including a food intake schedule for each of eggs and milk is generated.

For eggs, each 100 grams of egg white contains approximately 11 grams of egg protein, and since different cooking forms denature the protein differently due to heat and soft-boiled eggs are more allergenic than hard-boiled eggs, it may be safer to proceed with hard-boiled eggs. Milk contains approximately 3 grams of milk protein per 100 grams of raw milk, and it may be safer to proceed with heated milk because heat denatures the protein and makes it less allergenic, unlike raw milk. As such, the oral immunotherapy guideline information may also include intake form information that it is safe to proceed with hard-boiled eggs or heated milk.

In addition, when administering oral immunotherapy for two foods, the oral immunotherapy guideline information is generated to ensure that the other food is taken at least two hours after one food. If an adverse event occurs to one food, the oral immunotherapy guideline information is generated such that the patient does not take the other food that day.

The starting dose of oral immunotherapy is determined with reference to the results of the oral food challenge (OFC) included in the patient information. That is, the starting dose of oral immunotherapy is determined to ⅓ to ½ of the last dose in which an adverse event occurs depending on the results of the oral food challenge.

The oral immunotherapy guideline information is generated so as to include the start date of an oral immunotherapy step, which is divided into initial escalation, buildup, and maintenance, and a food intake schedule including the amount of food corresponding to the amount of protein to be taken per day.

In the initial escalation step, a food intake schedule, in which the starting dose of oral immunotherapy is maintained for one to two weeks, the dose of oral immunotherapy is increased by 0.001 grams every week until a food antigen protein level of 0.01 grams is reached, the dose of oral immunotherapy is maintained for one to two weeks when a food antigen protein level of 0.01 grams is reached, the dose of oral immunotherapy is increased by 0.01 grams every week until a food antigen protein level of 0.05 grams is reached, the dose of oral immunotherapy is maintained for three to seven days when a food antigen protein level of 0.05 grams is reached, and the dose of oral immunotherapy is increased by 0.01 grams for three to seven days until a food antigen protein level of 0.1 grams is reached, is generated.

In the initial escalation step, dosage information for daily dosing of leukotriene receptor antagonist (LTRA) in order to reduce the risk of allergic reactions is included in the oral immunotherapy guideline information.

The buildup step, in which a food antigen protein level of 0.1 grams has been reached, takes at least 22 weeks, and dosage information for daily dosing of leukotriene receptor antagonist if there is regular administration during first 2 months of buildup is included in the oral immunotherapy guideline information.

The leukotriene receptor antagonist may be administrated alone or in combination with antihistamine. In addition, antihistamine may be administered alone in order to reduce the risk of allergic reactions during initial escalation and during buildup.

In the buildup step, a daily buildup schedule, in which food is taken while a dose equivalent to 3 to 5% of the dose of the previous day is continuously increased every day until a food antigen protein level of 4 grams is reached, is generated, as shown in graph (a) of FIG. 5. The daily buildup schedule is generated such that each dose is built up after at least one day of confirmation of no adverse event at each dose, and the same dose is taken on Friday, Saturday, and Sunday without buildup. Alternatively, the daily buildup schedule may be generated such that the dose is built up after at least one day of confirmation of no adverse event at each dose and the dose is also built up on Friday, Saturday, and Sunday.

Alternatively, in the buildup step, a weekly buildup schedule, in which a dose equivalent to 20 to 25% of the dose of the previous week is increased until a food antigen protein level of 4 grams is reached, the dose is maintained for one week, and a dose equivalent to 20 to 25% of the maintained dose is taken the next week, may be generated, as shown in graph (b) of FIG. 5. The weekly buildup schedule is generated such that the dose is built up after at least 7 days of confirmation of no adverse event at each dose.

The maintenance step, in which a food antigen protein level of 4 grams has been reached, takes at least one year, and a message to visit a clinic may be included in the oral immunotherapy guideline information since an oral food challenge is required when a food antigen protein level of 4 grams is reached. If an adverse event occurs as the result of the oral food challenge, the oral immunotherapy guideline information is generated so as to repeat the oral food challenge after 2 grams to 4 grams of food antigen protein is taken every day for 2 months. If no adverse event occurs as the result of the oral food challenge, the oral immunotherapy guideline information is generated so as to take 4 grams of food antigen protein once a day or at least four times every week.

The oral immunotherapy step may further include dietary restriction to limit protein intake after the maintenance. The dietary restriction is configured to evaluate whether the patient has reached “tolerance” which allows the patient to eat the food freely through oral immunotherapy and to determine whether the allergy does not return even if the patient does not eat the food consistently. After one-month dietary restriction, the oral immunotherapy guideline information may include a message to visit the clinic for the oral food challenge. If an adverse event occurs as the result of the oral food challenge, the oral immunotherapy guideline information is generated so as to repeat the same type of oral food challenge for one month after taking 2 to 4 grams of food antigen protein at least four times for 6 months. If no adverse event occurs as the result of the oral food challenge, oral immunotherapy is terminated.

Subsequently, the oral immunotherapy guideline information is transmitted to the patient terminal and displayed (S320). That is, the amount of food corresponding to the amount of protein to be taken per day from the start date of the oral immunotherapy step according to the food intake schedule included in the oral immunotherapy guideline information is displayed on the patient terminal 100 every day, as shown in FIG. 2B. In the buildup step, a food intake schedule, which is one of a daily buildup schedule and a weekly buildup schedule, is selected according to a selection command input to any one of the patient terminal 100 and the medical staff terminal 200. According to the selected food intake schedule, the amount of food corresponding to the amount of protein to be taken per day is displayed on the patient terminal 100 every day.

The oral immunotherapy guideline information displayed on the patient terminal 100 also includes a message instructing the patient to take the food including the protein dose to be taken per day once a day, at a certain time of day, immediately after a meal, with preparation of a standby medicine for allergy symptoms, and a message instructing the patient to refrain from active movement for two hours after intake.

When the symptom information about the symptoms after intake of the protein is input to the patient terminal 100, the symptom information is transmitted to the medical staff terminal 200 (S330). The symptom information includes text information recorded on the patient terminal 100, image information of the area where the symptom occurs, and voice information of the recorded patient's voice. In addition, the amount of protein actually consumed by the patient, the date and time of intake, and medication history information about the type, time, and dose of the medication administered are also input to the patient terminal 100 and transmitted to the medical staff terminal 200.

If acute infection symptoms, such as fever, vomiting, or diarrhea, occur during oral immunotherapy, the food intake schedule included in the oral immunotherapy guideline information is modified such that protein intake is stopped for 3 days, ¼ of the last intake is taken on the 4th day, ½ of the last intake is taken on the 5th day, and the last intake is taken on the 6th day and subsequent days.

The symptom information is analyzed by the medical staff terminal 200 to determine an adverse event grade (S340). After the received symptom information is analyzed, a CoFAR grade of 1 is determined if the symptoms in one of the skin, conjunctival, upper respiratory tract, and digestive system categories are all mild, a CoFAR grade of 2 is determined if even any one of the symptoms in one of the skin, conjunctival, upper respiratory tract, and digestive system categories is severe or if the symptoms in two or more categories are all mild, and a CoFAR grade of 3 is determined if severe abdominal pain occurs or vomiting/diarrhea occurs three or more times or if the symptoms in the lower respiratory tract category are all mild. In this case, the cough is moderate or severe. A CoFAR grade of 4 is determined if any one of the symptoms in the lower respiratory tract category is severe or if any one of the symptoms in the circulatory system category occurs, and a CoFAR grade of 5 is determined for death. CoFAR grade 1 may be categorized as mild, CoFAR grade 2 as moderate, and CoFAR grade 3 or higher as severe, or CoFAR grade 1 as mild, CoFAR grades 2 and 3 as moderate, and

CoFAR grade 4 or higher as severe.

The oral immunotherapy guideline information is modified according to the adverse event grade and is transmitted to the patient terminal 100 for display (S350). When a daily buildup schedule is selected in the buildup step, the treatment effect is the same as when a weekly buildup schedule is selected, but there are differences in how to respond to adverse events according to the grade.

First, when the daily buildup schedule is selected, if the adverse event is determined to be mild (CoFAR grade 1), the oral immunotherapy guideline information is modified so as to increase the amount of the protein intake according to the daily buildup schedule after determining that the patient is symptom-free when taking the same amount of antihistamine the next day after intake of antihistamine and is transmitted to the patient terminal 100 for display. Here, the oral immunotherapy guideline information may be modified so as to take steroid if there is no improvement within 5 to 30 minutes after taking the antihistamine.

For example, as shown in Table 1, if 0.105 grams of egg protein is taken and an adverse event occurs, the food intake schedule is modified so as to increase the amount of egg protein after the next day if no adverse event occurs after the same 0.105 grams of egg protein is taken the next day.

TABLE 1

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.105 | 0.105 | 0.11 | 0.115 | 0.115 | 0.12 |
| intake | | | | | | | |
| (g) | | | | | | | |
| Increment | | 0.005 | 0 | 0.005 | 0.005 | 0 | 0.005 |
| (g) | | | | | | | |
| Symptom | X | ◯ | X | X | ◯ | X | X |

If 0.105 grams of egg protein causes an adverse event for three days, as shown in Table 2 below, the food intake schedule is modified so as to take 0.105 grams of egg protein for 7 days and increase the amount of egg protein after confirming that there are no symptoms.

TABLE 2

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.105 | 0.105 | 0.105 | 0.105 | 0.105 | 0.105 |
| intake | | | | | | | |
| (g) | | | | | | | |
| Increment | | 0.005 | 0 | 0 | 0 | 0 | 0 |
| (g) | | | | | | | |
| Symptom | X | ◯ | ◯ | ◯ | ◯ | X | X |
| Date | 8 | 9 | 10 | 11 | 12 | 13 | 14 |
| Protein | 0.105 | 0.105 | 0.105 | 0.105 | 0.105 | 0.11 | 0.115 |
| intake | | | | | | | |
| (g) | | | | | | | |
| Increment | 0 | 0 | 0 | 0 | 0 | 0.005 | 0.005 |
| (g) | | | | | | | |
| Symptom | X | X | X | X | X | X | X |

Here, buildup failure is defined as an adverse event occurring again when buildup is attempted after maintaining the same amount for 7 days and confirming no symptoms, and an alert message indicating that a consultation with the medical staff is required is transmitted to the patient terminal 100 for display if buildup failure is repeated twice or more.

If the adverse event is determined to be moderate (CoFAR grade 2), the food intake schedule is modified so as to take both antihistamine and steroid, to maintain the dose taken on the day immediately preceding the date of the adverse event for 7 days, and to increase the amount of protein, as shown in Table 3.

TABLE 3

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.105 | 0.1 | 0.1 | 0.1 | 0.1 | 0.1 |
| intake | | | | | | | |
| (g) | | | | | | | |
| Increment | | 0.005 | 0 | 0 | 0 | 0 | 0 |
| (g) | | | | | | | |
| Symptom | X | ◯ | X | X | X | X | X |

| Date | 8 | 9 | 10 | 11 | 12 | 13 | 14 |
|---|---|---|---|---|---|---|---|
| Protein | 0.1 | 0.1 | 0.105 | 0.11 | 0.115 | 0.12 | 0.125 |
| intake | | | | | | | |
| (g) | | | | | | | |
| Increment | 0 | 0 | 0.005 | 0.005 | 0.005 | 0.005 | 0.005 |
| (g) | | | | | | | |
| Symptom | X | X | X | X | X | X | X |

Here, buildup failure is defined as an adverse event occurring again when buildup is attempted after maintaining the same amount for 7 days and confirming no symptoms, and an alert message indicating that a consultation with the medical staff is required is transmitted to the patient terminal 100 for display if buildup failure is repeated twice or more.

If the adverse event is determined to be severe (CoFAR grade 3 or higher), a warning message indicating that there is a need to immediately stop oral immunotherapy and to consult the medical staff is transmitted to the patient terminal 100 for display. In addition, the oral immunotherapy guideline information is modified so as to take antihistamine and steroid and to also use inhaled medication, if any, and is transmitted to the patient terminal 100 for display.

When the weekly buildup schedule is selected, if the adverse event is determined to be mild (CoFAR grade 1), the oral immunotherapy guideline information is modified so as to increase the amount of protein intake according to the weekly buildup schedule after the antihistamine is taken and the same amount is taken the next day to confirm that the patient is symptom free, and is transmitted to the patient terminal 100 for display. Here, the oral immunotherapy guideline information may be modified so as to take steroid if there is no improvement within 5 to 30 minutes after taking the antihistamine.

For example, as shown in Table 4, if 0.125 grams of egg protein is taken and an adverse event occurs, the food intake schedule is modified such that the same 0.125 grams of egg protein is taken the next day, if no adverse event occurs, the dose is taken for a total of seven days, and if it is confirmed that no adverse events occur, the amount of protein is increased.

TABLE 4

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.125 | 0.125 | 0.125 | 0.125 | 0.125 | 0.125 |
| intake | | | | | | | |
| (g) | | | | | | | |
| Increment | | 0.025 | 0 | 0 | 0 | 0 | 0 |
| (g) | | | | | | | |
| Symptom | X | ◯ | X | X | X | X | X |
| Date | 8 | 9 | 10 | 11 | 12 | 13 | 14 |
| Protein | 0.125 | 0.125 | 0.15 | 0.15 | 0.15 | 0.15 | 0.15 |
| intake | | | | | | | |
| (g) | | | | | | | |
| Increment | 0 | 0 | 0.025 | 0 | 0 | 0 | 0 |
| (g) | | | | | | | |
| Symptom | X | X | X | X | X | X | X |

Here, buildup failure is defined as an adverse event occurring again when buildup is attempted after maintaining the same amount for 7 days and confirming no symptoms, and an alert message indicating that a consultation with the medical staff is required is transmitted to the patient terminal 100 for display if buildup failure is repeated twice or more.

As shown in Table 5 below, if 0.125 grams of egg protein is taken and an adverse event occurs for three days, the food intake schedule is modified such that 0.125 grams of egg protein is taken the next day, if no adverse event occurs, 0.125 grams of egg protein is taken for a total of seven days, and if it is confirmed that no adverse events occur, the amount of egg protein is increased.

TABLE 5

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.125 | 0.125 | 0.125 | 0.125 | 0.125 | 0.125 |
| intake | | | | | | | |
| (g) | | | | | | | |
| Increment | | 0.025 | 0 | 0 | 0 | 0 | 0 |
| (g) | | | | | | | |
| Symptom | X | X | X | ◯ | X | X | X |
| Date | 8 | 9 | 10 | 11 | 12 | 13 | 14 |
| Protein | 0.125 | 0.125 | 0.15 | 0.15 | 0.15 | 0.15 | 0.15 |
| intake | | | | | | | |
| (g) | | | | | | | |
| Increment | 0 | 0 | 0.025 | 0 | 0 | 0 | 0 |
| (g) | | | | | | | |
| Symptom | X | X | X | X | X | X | X |

Here, buildup failure is defined as an adverse event occurring again when buildup is attempted after maintaining the same amount for 7 days and confirming no symptoms, and an alert message indicating that a consultation with the medical staff is required is transmitted to the patient terminal 100 for display if buildup failure is repeated twice or more.

If the adverse event is determined to be moderate (CoFAR grade 2), the food intake schedule is modified so as to take both antihistamine and steroid, to maintain the dose taken on the day immediately preceding the date of the adverse event for 7 days, and to increase the amount of protein, as shown in Table 6.

TABLE 6

| | Date | | | | | | |
|---|---|---|---|---|---|---|---|
| | Sun | Mon | Tue | Wed | Thu | Fri | Sat |
| | 1 | 2 | 3 | 4 | 5 | 6 | 7 |
| Protein | 0.1 | 0.125 | 0.1 | 0.1 | 0.1 | 0.1 | 0.1 |
| intake | | | | | | | |
| (g) | | | | | | | |
| Increment | | 0.025 | 0 | 0 | 0 | 0 | 0 |
| (g) | | | | | | | |
| Symptom | X | ◯ | X | X | X | X | X |

| Date | 8 | 9 | 10 | 11 | 12 | 13 | 14 |
|---|---|---|---|---|---|---|---|
| Protein | 0.1 | 0.1 | 0.125 | 0.125 | 0.125 | 0.125 | 0.125 |
| intake | | | | | | | |
| (g) | | | | | | | |
| Increment | 0 | 0 | 0.025 | 0 | 0 | 0 | 0 |
| (g) | | | | | | | |
| Symptom | X | X | X | X | X | X | X |

Here, buildup failure is defined as an adverse event occurring again when buildup is attempted after maintaining the same amount for 7 days and confirming no symptoms, and an alert message indicating that a consultation with the medical staff is required is transmitted to the patient terminal 100 for display if buildup failure is repeated twice or more.

If the adverse event is determined to be severe (CoFAR grade 3 or higher), a warning message indicating that there is a need to immediately stop oral immunotherapy and to consult the medical staff is transmitted to the patient terminal 100 for display. In addition, the oral immunotherapy guideline information is modified so as to take antihistamine and steroid and to also use inhaled medication, if any, and is transmitted to the patient terminal 100 for display.

In step S330, the medical staff terminal 200 also receives the amount of food antigen protein actually consumed by the patient, the date and time of intake, and medication history information about the type, time, and dose of medication administered, and if it is determined that food has not taken according to the oral immunotherapy guideline information, a message indicating that termination of oral immunotherapy and a consultation with the medical staff are required is transmitted to the patient terminal 100 for display.

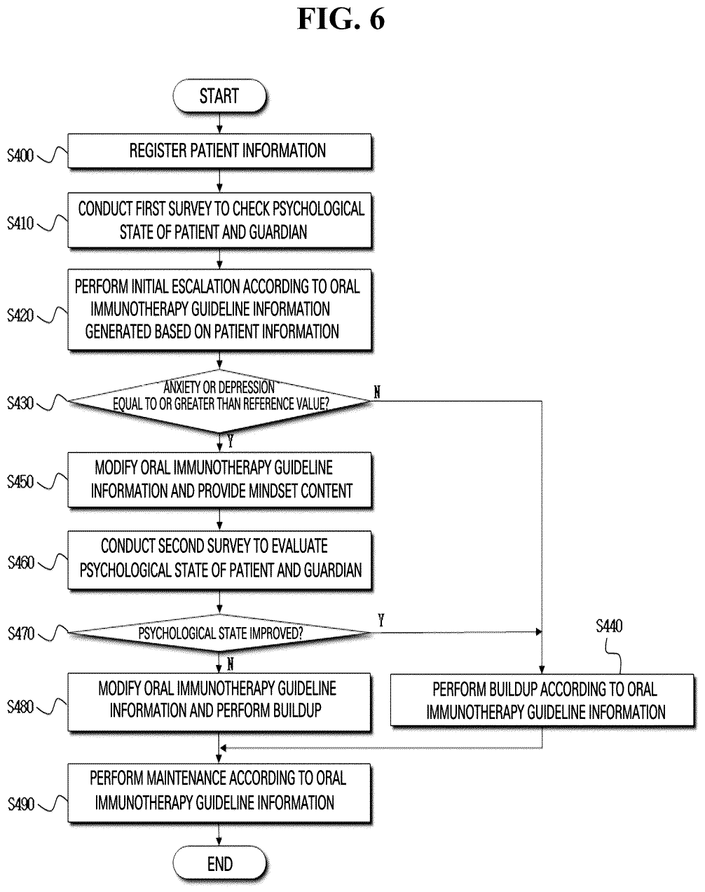

FIG. 6 is a flowchart describing a food allergy treatment service provision method using oral immunotherapy according to another embodiment of the present invention.

Referring to FIG. 6, first, the patient information input to the patient terminal 100 is transmitted to the medical staff terminal 200 for registration (S400). The patient information includes at least one of the patient's age, gender, underlying diseases, medication, allergenic foods, past medical history, and oral food challenge (OFC) results.

In accordance with an embodiment, the patient information may include additional patient test results, including a psychological questionnaire for the patient, a psychological questionnaire for the guardian, the presence or absence of specific IgE on a blood test of the patient, and the results of analysis of the gut microbiome. The psychological questionnaire may correspond to anxiety and quality of life assessment tools (e.g., Korean version of the Beck anxiety inventory (K-BAI) and the food allergy anxiety scale for patients (SOFAA-C or FAAS-C) and for guardians (SOFAA-P)), patient health questionnaire (e.g., Korean version of the patient health questionnaire PHQ-9), a resilience scale test (e.g., the Davidson resilience scale, K-CD-RISC), Korean personality rating scale for children (KPRC), food allergy quality of life-parental burden, Korean children's eating behavior questionnaire (K-CEBQ), etc.

Based the results of the patient's psychological questionnaire and/or the guardian's psychological questionnaire, the patient terminal 100 may modify the guideline information for buildup or may add a procedure for providing mindset content for psychological reassurance. For example, the mindset content may provide therapy content, such as psychological counseling or educational videos on oral immunotherapy, in a specific step. For example, the supportive therapy content may include videos including images of immune improvement in the patient and patients with side effects and data about treatment outcome rates (reaching desensitization) of patients with similar symptoms to the patient. That is, the patient terminal 100 may collect data about similar patients based on the input symptom information. Alternatively, the content may include encouragement messages from patients who have had similar experiences (allergic reactions, side effects, etc.) and guardians and reviews of treatment outcome. In this case, the patient terminal 100 may form the content by collecting appropriate data based on the patient information, the buildup step, etc. among the previously collected patient data.

In addition, it is possible to provide a supplementary therapy of deficient strain based on the results of analysis of the microbiome.

Also, in accordance with an embodiment, different kinds of guideline information may be provided for different steps based on gender and age. For example, if the patient is a male over the age of 6, the mindset content may be further provided. Alternatively, symptom information that is input may be provided to the medical staff terminal 200, and the medical staff terminal 200 may provide additional feedback information to the patient terminal 100.

In accordance with an embodiment, a first survey to check the psychological state of the patient and the guardian is conducted through the patient terminal 100 and the medical staff terminal 200 (S410). The first survey is conducted with reference to the Korean version of the patient health questionnaire-9 (PHQ-9). That is, for the first survey, question information generated by the medical staff terminal 200 with reference to the Korean version of the patient health questionnaire-9 is transmitted to the patient terminal 100. When answer information for the first survey is input to the patient terminal 100, the answer information is transmitted to the medical staff terminal 200 for analysis. The patient terminal 100 may change the timing and cycle of buildup in the oral immunotherapy guideline information according to the analyzed results of the first survey.

The patient terminal 100 proceeds with initial escalation according to the oral immunotherapy guideline information generated based on the patient information (S420). That is, the oral immunotherapy guideline information is generated based on the patient information registered in the medical staff terminal 200 and transmitted to the patient terminal 100 during initial escalation. The oral immunotherapy guideline information is generated so as to include the start date of an oral immunotherapy step, which is divided into initial escalation, buildup, and maintenance, and a food intake schedule including the amount of food corresponding to the amount of protein to be taken per day.

At this time, the starting dose of oral immunotherapy is determined with reference to the results of the oral food challenge (OFC) included in the patient information. That is, the starting dose of oral immunotherapy is determined to ⅓ to ½ of the last dose upon which an adverse event occurs depending on the results of the oral food challenge.

Specifically, in the initial escalation step, a food intake schedule, in which the starting dose of oral immunotherapy is maintained for one to two weeks, the dose of oral immunotherapy is increased by 0.001 grams every week until a food antigen protein level of 0.01 grams is reached, the dose of oral immunotherapy is maintained for one to two weeks when a food antigen protein level of 0.01 grams is reached, the dose of oral immunotherapy is increased by 0.01 grams every week until a food antigen protein level of 0.05 grams is reached, the dose of oral immunotherapy is maintained for three to seven days when a food antigen protein level of 0.05 grams is reached, and the dose of oral immunotherapy is increased by 0.01 grams for three to seven days until a food antigen protein level of 0.1 grams is reached, is generated. Also, in the initial escalation step, dosage information for daily dosing of leukotriene receptor antagonist (LTRA) in order to reduce the risk of allergic reactions is included in the oral immunotherapy guideline information. The leukotriene receptor antagonist may be administrated alone or in combination with antihistamine. In addition, antihistamine may be administered alone in order to reduce the risk of allergic reactions during initial escalation.

Upon determining that anxiety and depression are less than a reference value from the results of analysis of the first survey on the medical staff terminal 200 (S430—N), the amount of food corresponding to the protein dose to be taken during buildup is transmitted to the patient terminal 100 for display every day such that buildup progresses according to the oral immunotherapy guideline information (S440).

The buildup step, in which a food antigen protein level of 0.1 grams has been reached, takes at least 22 weeks, and dosage information for daily dosing of leukotriene receptor antagonist during first 2 months of buildup is included in the oral immunotherapy guideline information. The leukotriene receptor antagonist may be administrated alone or in combination with antihistamine. In addition, antihistamine may be administered alone in order to reduce the risk of allergic reactions during buildup.

In the buildup step, a daily buildup schedule, in which food is taken while a dose equivalent to 3 to 5% of the dose of the previous day is continuously increased every day until a food antigen protein level of 4 grams is reached, is generated, as shown in graph (a) of FIG. 5. The daily buildup schedule is generated such that each dose is built up after at least one day of confirmation of no adverse event at each dose, and the same dose is taken on Friday, Saturday, and Sunday without buildup. Alternatively, in the buildup step, a weekly buildup schedule, in which a dose equivalent to 20 to 25% of the dose of the previous week is increased until a food antigen protein level of 4 grams is reached, the dose is maintained for one week, and a dose equivalent to 20 to 25% of the maintained dose is taken the next week, may be generated, as shown in graph (b) of FIG. 5. The weekly buildup schedule is generated such that the dose is built up after at least 7 days of confirmation of no adverse event at each dose.

Upon determining that at least one of anxiety and depression is equal to or greater than the reference value from the results of analysis of the first survey on the medical staff terminal 200 (S430—Y), the oral immunotherapy guideline information is modified so as to take the same dose of protein for a predetermined period of time and transmitted to the patient terminal 100 (S450). That is, the oral immunotherapy guideline information is modified so as to take the same dose, i.e. 0.1 grams, of protein for one to two months without proceeding with the buildup after completion of the initial escalation and is displayed on the patient terminal 100.

In this case, guideline may be provided so as not to proceed with the buildup even if no symptom information is input.

In addition, in step S450, the same amount of protein is taken for a predetermined period of time while providing mindset content for psychological stability. That is, psychological counseling, supportive therapy, watching educational videos, watching videos for mindset, etc. are applied to the patient and guardian such that the guardian patient and can find psychological stability. In addition, a supplementary therapy of deficient strain is provided to the patient based on the results of analysis of the microbiome. At this time, different kinds of guideline information may be provided for different steps based on gender and age.

Subsequently, a second survey to evaluate the psychological state of the patient and the guardian is conducted (S460). That is, question information for the second survey is generated on the medical staff terminal 200 and transmitted to the patient terminal 100, and when answer information for the second survey is input to the patient terminal 100, the answer information is transmitted to the medical staff terminal 200 for analysis.

The second survey is conducted based on question information generated with reference to the Korean version of the Conner-Davidson resilience scale (CD-RISC), which is a tool for measuring self-resilience, the Korean version of the Beck anxiety inventory (K-BAI), the Korean personality rating scale for children (KPRC), Children's Eating Behavior Questionnaire (K-CEBQ), and food allergy quality of life-parental burden.

In addition, in step S460, the patient's blood and gut microbiome test results may be input to the medical staff terminal 200 and analyzed in conjunction with answer information to the second survey. For example, the test is preferably performed 2-6 months after initiation of treatment. The blood test is performed by separating serum and measuring total IgE (Immunoglobulin E) and specific IgE and IgG4 (Immunoglobulin G4) with ImmunoCAP.

Upon determining that the psychological state has improved from the result of analysis of the second survey on the medical staff terminal 200 (S470—Y), the amount of food corresponding to the protein dose to be taken during buildup is transmitted to the patient terminal 100 for display every day such that the buildup proceeds according to the oral immunotherapy guideline information (S440).

Although the use of gut microbiome testing to evaluate whether the psychological state has improved is illustrated here, gut microbiome testing may be utilized not only to evaluate whether the psychological state has improved but also to generate an immunologically conducive gut environment or to reduce various side effects therethrough.

On the other hand, upon determining that the psychological state has not improved (S470—N), the oral immunotherapy guideline information is modified and the buildup proceeds (S480). For example, if the buildup step is set to a daily buildup schedule, the buildup step is changed to a weekly buildup schedule, if the buildup step is set to a weekly buildup schedule, the buildup step is changed to a daily buildup schedule, and the modified oral immunotherapy guideline information is transmitted to the patient terminal 100 for display during buildup.

Also, upon determining that the psychological state has not improved, the content such as the psychological counseling or the educational videos may be provided.

Alternatively, in the buildup step, if the daily buildup schedule is set such that food is taken while a dose equivalent to 5% of the dose of the previous day is continuously increased every day until a food antigen protein level of 4 grams is reached, the dose is reduced to less than 5% (e.g., 2.5%), if the weekly buildup schedule is set to increase the dose by 25% from the previous week until a food antigen protein level of 4 grams is reached, the dose is reduced to less than 25% (e.g., 10%), and the modified oral immunotherapy guideline information is transmitted to the patient terminal 100 for display during buildup.

Subsequently, the maintenance proceeds according to the oral immunotherapy guideline information (S490). The maintenance step, in which a food antigen protein level of 4 grams has been reached, takes at least one year, and a message to visit a clinic may be included in the oral immunotherapy guideline information since an oral food challenge is required when a food antigen protein level of 4 grams is reached. If an adverse event occurs as the result the oral food challenge, the oral immunotherapy guideline information is generated so as to repeat the oral food challenge after 2 grams to 4 grams of food antigen protein is taken every day for 2 months. If no adverse event occurs as the result of the oral food challenge, the oral immunotherapy guideline information is generated so as to take 4 grams of food antigen protein once a day or at least four times every week.

The oral immunotherapy step may further include dietary restriction to limit protein intake after the maintenance. The dietary restriction is configured to evaluate whether the patient has reached “tolerance” which allows the patient to eat the food freely through oral immunotherapy and to determine whether the allergy does not return even if the patient does not eat the food consistently. After one-month dietary restriction, the oral immunotherapy guideline information may include a message to visit the clinic for the oral food challenge. If an adverse event occurs as the result of the oral food challenge, the oral immunotherapy guideline information is generated so as to repeat the same type of oral food challenge for one month after taking 2 to 4 grams of food antigen protein at least four times for 6 months. If no adverse event occurs as the result of the oral food challenge, oral immunotherapy is terminated.

The oral immunotherapy guideline information displayed on the patient terminal 100 also includes a message instructing the patient to take the food including the protein dose to be taken per day once a day, at a certain time of the day, immediately after a meal, with a standby medicine for allergy symptoms, and a message instructing the patient to refrain from active movement for two hours after intake.

When the symptom information about the symptoms after intake of the protein is input to the patient terminal 100 during oral immunotherapy, the symptom information is transmitted to the medical staff terminal 200. The symptom information includes text information recorded on the patient terminal 100, image information of the area where the symptom occurs, and voice information of the recorded patient's voice. In addition, the amount of protein actually consumed by the patient, the date and time of intake, and medication history information about the type, time, and dose of the medication administered are also input to the patient terminal 100 and transmitted to the medical staff terminal 200.

If acute infection symptoms, such as fever, vomiting, or diarrhea, occur during oral immunotherapy, the food intake schedule included in the oral immunotherapy guideline information is modified such that protein intake is stopped for 3 days, ¼ of the last intake is taken on the 4th day, ½ of the last intake is taken on the 5th day, and the last intake is taken on the 6th day and subsequent days.

The symptom information is analyzed by the medical staff terminal 200 to determine an adverse event grade. After the received symptom information is analyzed, a CoFAR grade of 1 is determined if the symptoms in one of the skin, conjunctival, upper respiratory tract, and digestive system categories are all mild, a CoFAR grade of 2 is determined if even any one of the symptoms in one of the skin, conjunctival, upper respiratory tract, and digestive system categories is severe or if the symptoms in two or more categories are all mild, and a CoFAR grade of 3 is determined if severe abdominal pain occurs or vomiting/diarrhea occurs three or more times or if the symptoms in the lower respiratory tract category are all mild. In this case, the cough is moderate or severe. A CoFAR grade of 4 is determined if even any one of the symptoms in the lower respiratory tract category is severe or if even any one of the symptoms in the circulatory system category occurs, and a CoFAR grade of 5 is determined for death. CoFAR grade 1 may be categorized as mild, CoFAR grade 2 as moderate, and CoFAR grade 3 or higher as severe, or CoFAR grade 1 as mild, CoFAR grades 2 and 3 as moderate, and CoFAR grade 4 or higher as severe.

The oral immunotherapy guideline information is modified according to the adverse event grade and is transmitted to the patient terminal 100 for display. When a daily buildup schedule is selected in the buildup step, the treatment effect is the same as when a weekly buildup schedule is selected, but there are differences in how to respond to adverse events according to the grade.

First, when the daily buildup schedule is selected, if the adverse event is determined to be mild (CoFAR grade 1), the oral immunotherapy guideline information is modified so as to increase the amount of the protein intake according to the daily buildup schedule after determining that the patient is symptom-free when taking the same amount of antihistamine the next day after intake of antihistamine and is transmitted to the patient terminal 100 for display. Here, the oral immunotherapy guideline information may be modified so as to take steroid if there is no improvement within 5 to 30 minutes after taking the antihistamine.

When the weekly buildup schedule is selected, if the adverse event is determined to be mild (CoFAR grade 1), the oral immunotherapy guideline information is modified so as to increase the amount of the protein intake according to the weekly buildup schedule after determining that the patient is symptom-free when taking the same amount of antihistamine the next day after intake of antihistamine and is transmitted to the patient terminal 100 for display. Here, the oral immunotherapy guideline information may be modified so as to take the steroid if there is no improvement within 5 to 30 minutes after taking the antihistamine.

The medical staff terminal 200 also receives the amount of protein actually consumed by the patient, the date and time of intake, and medication history information about the type, time, and dose of medication administered, and if it is determined that food has not t consumed according to the oral immunotherapy guideline information, a message indicating that termination of oral immunotherapy and a consultation with the medical staff are required is transmitted to the patient terminal 100 for display.

Some of the steps performed by the medical staff terminal 200 may be performed by the patient terminal 100, and the patient terminal 100 may modify or generate guideline information through data received from the medical staff terminal 200.

The foregoing description of the present invention is for illustrative purposes only, and a person having ordinary skill in the art to which the present invention pertains will understand that the present invention can be easily modified to other specific forms without changing the technical idea or essential features of the present invention. It should therefore be understood that the embodiments described above are illustrative, not restrictive, in all respects. For example, each component described in a single form may also be implemented in a distributed manner, and similarly, components described as being distributed may also be implemented in a combined form.

The scope of the present invention is defined by the appended claims, rather than by the detailed description, and should be construed as including the meaning and scope of the claims and all modifications or variations derived from the equivalents thereto.

1. A food allergy treatment service provision method using oral immunotherapy, the food allergy treatment service provision method comprising:
(a) transmitting patient information input to a patient terminal to a medical staff terminal connected to the patient terminal over a network;
(b) generating oral immunotherapy guideline information based on the patient information received by the medical staff terminal;
(c) transmitting the oral immunotherapy guideline information to the patient terminal for display;
(d) when symptom information about a symptom after protein intake is input to the patient terminal, transmitting the symptom information to the medical staff terminal;
(e) analyzing the symptom information on the medical staff terminal to determine an adverse event grade; and
(f) modifying the oral immunotherapy guideline information according to the adverse event grade and transmitting the modified oral immunotherapy guideline information to the patient terminal for display.

2. The food allergy treatment service provision method according to claim 1, wherein step (b) comprises determining a starting dose of oral immunotherapy with reference to a result of an oral food challenge (OFC) included in the patient information.

3. The food allergy treatment service provision method according to claim 2, wherein step (b) is generating the oral immunotherapy guideline information comprising a start date of an oral immunotherapy step, which is divided into initial escalation, buildup, and maintenance, and a food intake schedule comprising an amount of food corresponding to an amount of protein to be taken per day.

4. The food allergy treatment service provision method according to claim 3, wherein the oral immunotherapy guideline information comprises a food intake schedule in which the starting dose of oral immunotherapy is maintained for one to two weeks in the initial escalation step, the dose of oral immunotherapy is increased by 0.001 grams every week until a food antigen protein level of 0.01 grams is reached, the dose of oral immunotherapy is maintained for one to two weeks when a food antigen protein level of 0.01 grams is reached, the dose of oral immunotherapy is increased by 0.01 grams every week until a food antigen protein level of 0.05 grams is reached, the dose of oral immunotherapy is maintained for three to seven days when a food antigen protein level of 0.05 grams is reached, and the dose of oral immunotherapy is increased by 0.01 grams for three to seven days until a food antigen protein level of 0.1 grams is reached.

5. The food allergy treatment service provision method according to claim 3, wherein the oral immunotherapy guideline information comprises a food intake schedule, which is one of a daily buildup schedule in which food is taken while a dose equivalent to 3 to 5% of the dose of a previous day is continuously increased every day until a food antigen protein level of 4 grams is reached in the buildup step, in which a food antigen protein level of 0.1 grams has been reached, and a weekly buildup schedule in which a dose equivalent to 15 to 25% of the dose of a previous week is increased, the dose is maintained for one week, and a dose equivalent to 15 to 25% of the maintained dose is taken next week.

6. The food allergy treatment service provision method according to claim 5, wherein step (c) comprises daily displaying an amount of food corresponding to an amount of protein to be taken per day on the patient terminal every day according to a food intake schedule selected according to a selection command input to any one of the patient terminal and the medical staff terminal, which is one of the daily buildup schedule and the weekly buildup schedule.

7. The food allergy treatment service provision method according to claim 3, wherein the oral immunotherapy guideline information comprises a message to visit a clinic since the oral food challenge is required in the maintenance step, in which a food antigen protein level of 4 grams has been reached.

8. The food allergy treatment service provision method according to claim 7, wherein the oral immunotherapy guideline information is generated so as to repeat the oral food challenge after 2 grams to 4 grams of food antigen protein is taken every day for 2 months if an adverse event occurs as the result of the oral food challenge.

9. The food allergy treatment service provision method according to claim 7, wherein the oral immunotherapy guideline information is generated so as to take 4 grams of food antigen protein at least four times every week if no adverse event occurs as the result of the oral food challenge.

10. The food allergy treatment service provision method according to claim 1, wherein the patient information comprises at least one of patient's age, gender, underlying diseases, medication, allergenic foods, past medical history, and a result of an oral food challenge (OFC).

11. The food allergy treatment service provision method according to claim 10, wherein the oral immunotherapy guideline information is generated so as to comprise a food intake schedule including an amount of food corresponding to an amount of protein to be taken per day for each of the allergenic foods.

12. The food allergy treatment service provision method according to claim 1, wherein the symptom information comprises text information recorded on the patient terminal, image information of an area where the symptom occurs, and voice information of recorded patient's voice.

13. A computer program stored on a computer-readable storage medium to provide oral immunotherapy guideline information, the computer program comprising a command configured to enable a processor to perform:
(a) transmitting patient information input to a patient terminal to a medical staff terminal connected to the patient terminal over a network;
(b) generating oral immunotherapy guideline information based on the patient information received by the medical staff terminal;
(c) transmitting the oral immunotherapy guideline information to the patient terminal for display;
(d) when symptom information about a symptom after protein intake is input to the patient terminal, transmitting the symptom information to the medical staff terminal;
(e) analyzing the symptom information on the medical staff terminal to determine an adverse event grade; and
(f) modifying the oral immunotherapy guideline information according to the adverse event grade and transmitting the modified oral immunotherapy guideline information to the patient terminal for display.

14. The computer program according to claim 13, wherein the patient information comprises at least one of a psychological questionnaire for a patient, a psychological questionnaire for a guardian, presence or absence of specific IgE on a blood test of the patient, and a result of analysis of microbiome.

15. The computer program according to claim 14, wherein a timing and cycle of buildup in the oral immunotherapy guideline information are changed based on a result of at least one of the psychological questionnaire of the patient and the psychological questionnaire of the guardian.

16. The computer program according to claim 15, wherein each of the psychological questionnaire of the patient and the psychological questionnaire of the guardian comprises at least one of an anxiety and quality of life assessment tool, a patient health questionnaire, and a resilience scale test.

17. The computer program according to claim 14, wherein mindset content for psychological reassurance is provided based on a result of at least one of the psychological questionnaire of the patient and the psychological questionnaire of the guardian.

18. The computer program according to claim 17, wherein the mindset content comprises psychological counseling and educational videos on oral immunotherapy.

19. The computer program according to claim 18, further comprising:
collecting, by the patient terminal, data about patients having similar symptoms based on symptom information of a patient input thereto and a buildup step, wherein
the data comprises an encouragement message and a review of treatment outcome.

20. The computer program according to claim 14, further comprising providing a supplementary therapy of strain based on the result of analysis of the microbiome.

21. A food allergy treatment service provision method using oral immunotherapy, the food allergy treatment service provision method comprising:
(a) conducting a first survey to check a psychological state of a patient through a patient terminal and a medical staff terminal connected to the patient terminal over a network;
(b) generating oral immunotherapy guideline information based on patient information registered in the medical staff terminal and transmitting the generated oral immunotherapy guideline information to the patient terminal during initial escalation;
(c) upon determining that at least one of anxiety and depression is equal to or greater than a reference value from a result of analysis of the first survey on the medical staff terminal, modifying the oral immunotherapy guideline information so as to take the same dose of protein for a predetermined period of time and transmitting the modified oral immunotherapy guideline information to the patient terminal;
(d) conducting a second survey to check the psychological state through the patient terminal and the medical staff terminal; and
(e) upon determining that the psychological state has not improved from a result of analysis of the second survey on the medical staff terminal, modifying the oral immunotherapy guideline information and transmitting the modified oral immunotherapy guideline information to the patient terminal during buildup.

22. The food allergy treatment service provision method according to claim 21, further comprising:
inputting a result of a blood and gut microbiome test to the patient terminal and transmitting the result to the medical staff terminal, wherein
step (e) is further analyzing the result of the blood and gut microbiome test to determine whether the psychological state has improved.

23. The food allergy treatment service provision method according to claim 21, wherein step (e) is transmitting, to the patient terminal during buildup, the oral immunotherapy guideline information modified so as to alternately perform a daily buildup schedule in which food is taken while a dose equivalent to 3 to 5% of the dose of a previous day is continuously increased every day until a food antigen protein level of 4 grams is reached in the buildup step, in which a food antigen protein level of 0.1 grams has been reached, and a weekly buildup schedule in which a dose equivalent to 15 to 25% of the dose of a previous week is increased, the dose is maintained for one week, and a dose equivalent to 15 to 25% of the maintained dose is taken next week.

24. The food allergy treatment service provision method according to claim 21, wherein step (e) is transmitting, to the patient terminal during buildup, the oral immunotherapy guideline information modified through any one of a change to less than 5% if a daily buildup schedule is set such that food is taken while a dose equivalent to 5% of the dose of a previous day is continuously increased every day until a food antigen protein level of 4 grams is reached, in the buildup step, in which a food antigen protein level of 0.1 grams has been reached, and a change to less than 25% if a weekly buildup schedule is set to increase a dose by 25% from a previous week until a food antigen protein level of 4 grams is reached.

25. A computer program stored on a computer-readable storage medium to implement a food allergy treatment service provision method using oral immunotherapy, the computer program comprising a command configured to enable a processor to perform:
(a) conducting a first survey to check a psychological state of a patient through a patient terminal and a medical staff terminal connected to the patient terminal over a network;
(b) generating oral immunotherapy guideline information based on patient information registered in the medical staff terminal and transmitting the generated oral immunotherapy guideline information to the patient terminal during initial escalation;
(c) upon determining that at least one of anxiety and depression is equal to or greater than a reference value from a result of analysis of the first survey on the medical staff terminal, modifying the oral immunotherapy guideline information so as to take the same dose of protein for a predetermined period of time and transmitting the modified oral immunotherapy guideline information to the patient terminal;
(d) conducting a second survey to check the psychological state through the patient terminal and the medical staff terminal; and
(e) upon determining that the psychological state has not improved from a result of analysis of the second survey on the medical staff terminal, modifying the oral immunotherapy guideline information and transmitting the modified oral immunotherapy guideline information to the patient terminal during buildup.

26. A food allergy treatment service provision A food system using oral immunotherapy, the food allergy treatment service provision system comprising:
a patient terminal configured to allow patient information to be input thereto; and
a medical staff terminal configured to receive the patient information transmitted from the patient terminal and generating oral immunotherapy guideline information, wherein
the patient terminal displays the oral immunotherapy guideline information and allows symptom information about a symptom after protein intake to be input thereto, and
the medical staff receives terminal the symptom information transmitted from the patient terminal, analyzes the received symptom information to determine an adverse event grade, modifies the oral immunotherapy guideline information according to the adverse event grade, and transmits the modified oral immunotherapy guideline information to the patient terminal.

27. The food allergy treatment service provision system according to claim 26, wherein
the patient terminal displays question information for first and second surveys to check a psychological state of a patient and a guardian, and, when answer information for the first and second surveys is input, transmits the input answer information to the medical staff terminal, and
the medical staff terminal generates the question information for the first and second surveys, transmits the generated question information to the patient terminal, analyzes the answer information transmitted from the patient terminal, modifies the oral immunotherapy guideline information according to a degree of anxiety or depression of the patient and the guardian, and transmits the modified oral immunotherapy guideline information to the patient terminal.
